(19) United States
(12) Patent Application Publication
(10) Pub. No.: US 2024/0091413 A1
(43) Pub. Date: Mar. 21, 2024

(54) HYDROGEL MICROPARTICLE-BASED LUBRICANT

(71) Applicant: Likarda, LLC, Kansas City, MO (US)

(72) Inventors: Megan E. Hamilton, Kansas City, MO (US); Stephen Harrington, Kansas City, MO (US); Karthik Ramachandran, Kansas City, MO (US); Lisa Stehno-Bittel, Kansas City, MO (US)

(21) Appl. No.: 18/256,726

(22) PCT Filed: Dec. 9, 2021
(86) PCT No.: PCT/US2021/062586

§ 371 Date: Jun. 9, 2023

Related U.S. Application Data
(60) Provisional application No. 63/123,218, filed on Dec. 9, 2020.

(51) Int. Cl. A61L 27/52; A61L 27/18; A61L 27/20; A61L 27/56

(52) U.S. Cl. CPC A61L 27/52; A61L 27/18; A61L 27/20; A61L 27/56; A61L 2300/452; A61L 2400/06; A61L 2430/24; A61L 2430/34

(57) ABSTRACT

Methods for viscosupplementation in a joint or surgical site of a human or animal subject are disclosed along with unique materials for achieving such beneficial outcomes. The methods generally comprise introducing into the joint or surgical site a plurality of porous viscoelastic solid hydrogel microparticles having a size of greater than about 30 pm, and substantially free of any cells, tissue, or therapeutic compounds. Kits for viscosupplementation in a joint or surgical site of a human or animal subject are also disclosed. The kits comprise the plurality of porous viscoelastic solid hydrogel microparticles having a size of greater than about 30 pm, and substantially free of any cells, tissue, or therapeutic compounds, and instructions for administering the same.

### CROSS-REFERENCE TO RELATED APPLICATIONS

The present application claims the priority benefit of U.S. Provisional Patent Application Ser. No. 63/123,218, filed Dec. 9, 2020, entitled HYDROGEL MICROPARTICLE-BASED LUBRICANT, incorporated by reference in its entirety herein.

### BACKGROUND

### Field of Invention

The present invention relates to fully crosslinked microparticles made of a variety of hydrogels for use as joint or surgical lubricant.

### Description of the Field

Among the general U.S. population, joint pain is the leading source of chronic pain and disability. Descriptions of osteoarthritis (OA), along with attempts to manage the symptoms, have been found in ancient documents, yet today patients still suffer. Despite an annual price tag of >$27 billion, patients with OA are unsatisfied with their care due to chronic pain and poor sleep even with aggressive treatment and report a poor quality of life. Additional causes of joint injury or inflammation that can lead to chronic pain and loss of cartilage include (but are not limited to) avascular necrosis, bone cancer, hypothyroidism, lupus, sarcoidosis, scleroderma, trauma and medications such as steroids.

The current standard of care for joint pain and loss of cartilage consists of medication for pain, intra-articular (IA) injections of glucocorticoids or hyaluronic acid (HA), and eventually surgery, typically joint replacement. New treatments such as autologous chondrocyte implants have produced successful functional outcomes but are hampered by the need for a donor site and decreased chondrogenic potential in the elderly. HA remains the most common injectable agent used in joint treatment, and is available in several formats include a dilute liquid solution, semi-dilute solution, or an entangled or partially crosslinked solution exhibiting some gel-like behavior. Often, such products are incorrectly referred to as “hydrogels” even though they are typically in liquid, flowable form suitable for injection through a small gauge needle and not highly viscous nor do they demonstrate any kind of semi-solid behavior.

The primary goal of administering HA is to supplement synovial HA that is lost during chronic joint damage. While current products offer some relief for joint pain, once in the joint they are quickly degraded by endogenous enzymes. This quick degradation is due to the fact that the starting material is uncrosslinked or only partially crosslinked, leading to minimal structural integrity. Moreover, HA is naturally degraded by endogenous hyaluronidase, which is enhanced in inflamed tissue. Low molecular weight HA (10-500 kDa) is rapidly cleared from the joint with a half-life of only a few hours, while high molecular weight HA (>500 kDA) still only lasts for up to 9 days in the joint. The use of HA and other polymers as joint lubricants or surgical lubricants are widely utilized in both the human health and veterinary health fields.

### SUMMARY

The present disclosure describes the first approach of using fully crosslinked viscoelastic hydrogel microparticles which can be delivered into the intraarticular spaces of a joint as a joint lubricant to reduce joint pain and damage in human or veterinary applications. The microparticles can also be used for other medical or veterinary applications where lubrication would be beneficial, such as to reduce abrasion or friction between implanted medical devices (e.g., surgical mesh) and surrounding tissue, or as dermal fillers, and the like. In contrast to the current uncrosslinked or partially crosslinked suspensions, gels, or pastes, the crosslinked microparticles remain in the joint or other implantation area for a longer period of time, thus providing longer duration pain relief and less damage to the joint.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 contains images of microparticles of different shapes that can be injected into joints or other areas of the body as lubricants. A) shows spherical shapes, B) teardrops, C) ovals, D) random tubule shapes, E) a mixture of shapes.

FIG. 2 contains images of microparticles prior to and after physical stress. A) Prior to stress the microparticles made of methacrylated hyaluronic acid have a relatively consistent shape. B) After physical stress (passed through a small-gauge needle) the microparticle sizes are small and less regular, but they are still solid particles. C) The same is true of another hydrogel example, acrylated hyaluronic acid loaded with a fluorescent dye for better imaging. Again the particles are consistent in size and shape initially. D) After passing through the needle, the particles fracture into smaller fragments, but still maintain a physical structure as seen via the green fluorescent dye.

FIG. 3 contains microscopic images, captured on a BioTek Cytation 5 Imaging Multi-Mode Reader, illustrating the variety of sizes that can be made as crosslinked particles. A) Average diameters of 2040+21 μm, B) Average diameters of 1214+46 μm, C) Average diameters of 361+19μμm, D) Mixture of sizes.

FIG. 4 provides micrographs, captured on a BioTek Cytation 5 Imaging Multi-Mode, Reader, of different materials that can be used to make the microparticles—materials used for each particle type are labeled in the figure.

FIG. 5 is a graph of the change in swelling ratio over time of different microparticle formulations.

FIG. 6 describes the rheological characteristics of two examples hydrogels: (A) PEGDA and (B) AHA.

FIG. 7A shows imaging of the different diffusion characteristics of microparticles based on the initial starting chemistry. AHA microspheres (on the left) allow fluorescent dextran probes to enter the microparticle and after washing (3 min) there is still are still dextran probes left in the microparticle, as well as 30 minutes after washing. In contrast, PEGDA lets few of the same dextran probe into the microparticle and when rinsed it is clear that the dextran probe was only able to enter the outer region of the microparticle, but was not able to diffuse into the microparticle core.

FIG. 7B is a graph of the diffusion characteristics based upon Fluorescence Exclusion experiments of two example hydrogel formulations shown in FIG. 7A.

FIG. 7C is a graph of the diffusion characteristics based upon Relative Diffusion Rate for two example hydrogel formulations based on the example images shown in in FIG. 7A.

FIG. 8 is a schematic providing one example of how the microparticles can be modified after crosslinking with complimentary molecules. Fully crosslinked microparticles still have unbound reactive groups on the surface; in the example thiol (SH) moieties. By exposing the microparticles to a fluorescently-linked PEG-maleimide (MAL-CF350) the maleimide binds to the available thiol moieties (SH) and the microparticles fluoresce as shown in the image on the left.

If the microparticles are first exposed to a PEG-maleimide that does not fluoresce the surface thiols are now bound to that PEG and subsequent exposure to fluorescent PEG-maleimides will result in no fluorescence as shown in the image on the left.

FIG. 9 contains graphs depicting the size distribution (monodispersity) of the microparticles from two different batches.

FIG. 10 is an example of the in vitro degradation rate at 37° C. of microparticles made of different chemistries.

FIG. 11 contains images showing that the hydrogels can be fluorescently labeled to track their location and duration in the body. A) The micrograph illustrates microparticles made of HA using a procedure that results in a smaller particle diameter. B) The micrograph illustrates microparticles made of PEG with a larger diameter.

FIG. 12 is a graph illustrating the in vivo degradation of one of the microparticle formulations.

FIG. 13 contains images illustrating the biocompatibility of a number of different formulations of microparticles near or in the omentum of healthy rats.

FIG. 14 contains images of the increased cartilage production (red) and improved smoothness of the articular cartilage surface in a cartilage defect osteoarthritic animal model in rat knees that were injected with (A.) Vehicle control as compared to (B.) a joint with crosslinked microparticles. The arrows point to the location in the joint where new cartilage should be produced. The vehicle joint (A.) has no new cartilage at the surface and only minimal cartilage below the bony surface. In addition, the surface is not smooth. In contrast, the joint that received microparticles (B.) has regions of new cartilage along both bony surface (arrows) and a smoother surface.

FIG. 15A is an image from in vivo studies after destabilization of the medial meniscus osteoarthritic animal model in rat knees that were injected with the control vehicle.

FIG. 15B is an image showing increased cartilage production and improved smoothness of the articular cartilage in a destabilization of the medial meniscus osteoarthritic animal model in rat knees that were injected with crosslinked microparticles (as compared with the vehicle control in FIG. 15A).

FIG. 16 is a micrograph of thiolated HA microparticles that were stored at room temperature in water for over 4 years and then imaged, showing that there was no loss of structure in the microparticles.

FIG. 17A is micrograph, captured on a BioTek Cytation 5 Imaging Multi-Mode, Reader, showing microparticles (PEG-MAL) prior to freezing in liquid nitrogen.

FIG. 17B is an image of the PEG-MAL microparticles after freezing in liquid nitrogen and subsequent thawing, showing intact microparticles following the cryopreservation protocols.

### DETAILED DESCRIPTION

The microparticles comprise, consist essentially, or even consist of a 3-dimensional matrix of fully crosslinked hydrogel polymer compounds (covalent or strongly ionic crosslinks), such that the resulting microparticle body is characterized as a porous viscoelastic solid (hydrogel) wherein elastic deformation is reversible (i.e., the microparticle is semi-rigid, but resilient such that the microparticle can flex under load and returns to its original shape after the load is removed). Suitable hydrogel precursor compounds for use in forming the microparticles include hydrogel-forming polymers, oligomers, and/or monomers, and as such are capable of forming a crosslinked or network structure or matrix through covalent or strongly ionic crosslinking, wherein liquid may be retained, suspended, entrapped, and/or encapsulated within the interstitial spaces or pores of the resulting elastic gelled structure or matrix body (hydrogel).

The microparticles are 3-dimensional self-sustaining bodies meaning that they retain their particular shape without an external support structure once that shape is formed and are not susceptible to deformation or creep merely due to its own weight or gravity. In other words, the self-sustaining body is not permanently deformable, or flowable, like a jelly, putty, or paste, but is resilient, such that the matrix body may temporarily yield or deform under force, and unless fractured will return to the original shape upon removal of the force. The microparticles will typically have a surface-to-surface dimension (e.g., diameter in the case of roughly spherical bodies) of less than about 2,000 μm, preferably less than about 1,500 μm, but greater than about 30 μm, preferably greater than about 100 μm. The hydrogel microparticles can also be characterized as microspheres, microbeads, or hydrogel microparticles. Depending upon the polymer system used, the microparticles can generally be characterized by a Young's elastic modulus ranging from about 1,000 Pa to about 2.5 MPa (megapascal), more preferably about 10,000 Pa to about 2 MPa, more preferably from about 25,000 Pa to about 1 MPa, more preferably from about 50,000 Pa to about 0.5 MPa. Although resilient and having an elastic component, the microparticles, under sufficient application of force, may fracture or break into smaller pieces; however, such subsequent pieces would still comprise the 3-dimensional polymer matrix, albeit as smaller-sized bodies or fragments. In other words, the hydrogel microparticles are not shear thinning, nor is the matrix body itself susceptible to dissolution or dilution in a solvent system; although swelling of individual matrix bodies may occur depending upon the solvent system (i.e., as liquid moves into the interstitial spaces or pores of the matrix). In one or more embodiments, the hydrogel microparticles can be characterized as irreversible hydrogels, meaning that once fractured, the matrix crosslinks will not reform or otherwise recover or self-heal.

Exemplary polymer precursor compounds used for forming the microparticles include fast-gelling natural polymers that form strong matrices, such as alginate, as well as slow-gelling polymer precursors selected from the group consisting of branched or unbranched hyaluronic acid, branched or unbranched functionalized hyaluronic acid, branched or unbranched functionalized polyethylene glycol, hyaluronan, fibrin, chitosan, collagen, polylactic acid, poly(L-lactic acid), polylactic-co-glycolic acid, polycaprolactone, polyvinyl alcohol, and combinations thereof. The matrix can further comprise crosslinking agents crosslinked with polymer compounds, selected from the group consisting of dithiothreitol, branched or unbranched functionalized polyethylene glycol, dithiols, ethylene glycol bis-mercaptoacetate, and combinations thereof. Exemplary functionalized polyethylene glycols include, without limitation, polyethylene glycol dithiol, polyethylene glycol diacrylate, polyethylene glycol divinyl sulfone, polyethylene glycol dimaleimide, and combinations thereof. The matrix could be homogenous comprising one type of covalently crosslinked polymer backbone (with or without an additional crosslinker of a different polymer type). The matrix could also be heterogenous comprising a mixture of two or more polymer precursors, such as a combination of high (>500 kDA, preferably >100 kDA) and low (<100 kDA, preferably <50 kDa) molecular weight polymer precursors, and/or a mixture of two or more crosslinking agents. In general, the amount of polymer precursor included in the precursor solution will be less than 50% w/w. The amount will range from about 1% to about 50% w/w for high molecular weight (>500 kDa, preferably >100 kDa), multi-substituted polymers, such as hyaluronic acid, and from about 5% to about 50% w/w for lower molecular weight polymers (<100 kDa, preferably <50 kDa) such as PEGDA or PEGMAL.

Examples of reactive groups from different backbone chemistries that can be used to form a hydrogel with the appropriate crosslinkers are listed in the Table below. Some of the reactions are initiated by UV light, while others are a chemical reaction.

TABLE
Reactive Groups and Crosslinking Chemistry

| Reactive Polymer | | Crosslink |
|---|---|---|
| (precursor) | Crosslinker | Mechanism |
| MeHA (methacrylated HA) | PEG diacrylate (PEGDA) | UV |
| | self-reactive | UV |
| AHA (acrylated HA) | dithiothreitol (DTT), PEG dithiol | chemical |
| | PEGDA | UV |
| | self-reactive | UV |
| ThHA (thiolated HA) | PEGDA, PEG divinyl sulfone, PEG | chemical |
| | dimaleimide | |
| PHA (pentenoate HA) | DTT, PEG dithiol | UV |
| NorHA (norbornene HA) | DTT, PEG dithiol | UV |
| PEGDA | self-reactive | UV |
| PEG diacrylamide | self-reactive | UV |
| 4 or 8-arm PEGMAL | DTT, PEG dithiol | chemical |
| | self-reactive | UV |
| 4 or 8-arm PEG vinyl sulfone | DTT, PEG dithiol, ethylene glycol bis- | chemical |
| | mercaptoacetate (BMA) | |
| | self-reactive | UV |
| 4 or 8-arm PEG acrylate | DTT, PEG dithiol, BMA | chemical |
| | self-reactive | UV |
| Collagen-SH (thiolated | PEGDA | Chemical |
| collagen) | | |
| Alginate | ion catalyst | Chemical |

Additional exemplary precursor compounds include, without limitation, non-alginate polysaccharides, collagen/gelatin, chitosan, agarose, and the like. These precursor compounds can be branched polymers with multiple arms or unbranched/linear polymer chains of a single backbone. They can further be functionalized by attaching dyes for visual observation. A particularly preferred hydrogel precursor compound is hyaluronic acid, or a hyaluronic acid/PEG mixture. The precursor compound(s) can also be functionalized with various biological entities, such as chondrogenic growth factors including IFG-1 (insulin like growth factor-1), TGF-beta 1 (transforming growth factor-beta 1), BMP-2 (bone morphogenetic protein-2), GDF-5 (growth differentiation factor-5). Contrastingly, anti-inflammatory cytokines such as IL-10 (interleukin 10) and TNF-alpha (tumor necrosis factor alpha) could be added. Immuno-modulatory cytokines such as IFN-gamma (interferon gamma) could also be added to the precursor compounds at the time of hydrogel fabrication. These molecules would be bound to the hydrogel matrix as opposed to encapsulated in the interstitial voids for simple diffusion out of the microparticle. Contrastingly, non-therapeutic chemicals could be added to the precursor compounds including nanoparticles that would change the physical characteristics of the hydrogel for example to increase permeability or to allow for tracking of the hydrogel as shown in FIG. 11.

Biocompatible hydrogels are also particularly preferred, depending upon the designated end use of the hydrogel. As used herein, “biocompatible” means that it is not harmful to living tissue, and more specifically that it is not biologically or otherwise undesirable, in that it can be administered to a subject without excessive toxicity, irritation, or allergic or immunogenic response, and does not cause any undesirable biological effects or interact in a deleterious manner. Biocompatible hydrogels would be selected to minimize any adverse side effects in the subject, as would be well known to one of skill in the art. Additional optional ingredients that may be included with the hydrogel precursor include fibronectin, laminin, collagen, other components of the extracellular matrix, and the like, including synthetic versions thereof.

The crosslinking profile of the microparticles can be tuned by adjusting the molecular weight of the precursor compounds, as well as the selected crosslinker, crosslinking conditions, and crosslinking process. For example, “tighter” or faster crosslinks may be desired in some embodiments to achieve a smoother bead surface and/or stronger gel by limiting the ability of molecules to leech out of the droplet/core into the surrounding environment during crosslinking. This can be achieved both by increasing molecular weight of the precursor species (e.g., ˜≥40 kDa) and/or by decreasing the crosslinking time by adjusting the crosslinking chemistry or initiation method. Crosslinking can be carried out by various mechanisms depending upon the particular hydrogel precursor compound.

In general, one technique for preparing the microparticles is described in detail in U.S. Pat. No. 9,642,814, filed Jun. 3, 2015, incorporated by reference herein. The method involves preparing a hydrogel precursor solution consisting of the hydrogel precursor compound and a divalent cation (e.g., calcium, barium, strontium, and combinations thereof), dispersed or dissolved in a solvent system. The divalent cations are dispersed or dissolved in the solvent system along with the hydrogel precursor compound. The divalent cations should be included in the solution at a level of from about 0.025 moles/liter to about 0.25 moles/liter, based upon the total volume of the solution taken as 100%.

The hydrogel precursor solution can also include optional hydrogel crosslinking agents, catalysts, additives, media, nutrients, pH buffers, density modifying agents, viscosity modifying agents, or the like. The hydrogel precursor solution is then combined with alginate to initiate gelation of the alginate around the hydrogel precursor solution (via an “inside out” gelation process) to yield core/shell microparticles. Each core/shell microparticle comprises an alginate shell surrounding a liquid core, which comprises the hydrogel precursor solution. This generally involves dropwise addition of the precursor solution to an alginate bath, such as by generating/extruding droplets of the precursor solution that are dropped or sprayed into the alginate bath. The amount of alginate in the solution can be varied, but can range from about 0.1% to about 2.0% weight/volume, based upon the total volume of the solution taken as 100%. In general, the viscosity of the alginate solution should be less than the viscosity of the hydrogel precursor solution. The viscosity of an alginate solution depends upon the alginate concentration and average molecular weight of the alginate polymer (i.e., length of alginate molecules or number of monomer units in the chains), with longer chains resulting in higher viscosities at similar concentrations. In one or more embodiments, the viscosity of the alginate solution will range from about 1 to about 20 cP, and preferably from about 1 to about 4 cP at room temperature (˜20 to 25° C.). More specifically, the ratio of viscosity of the hydrogel precursor solution to the viscosity of the alginate solution should be greater than 1 at room temperature. In one more embodiments, the ratio of viscosity of the hydrogel precursor solution to the viscosity of the alginate solution is from about 1:1 to about 1000:1. In one or more embodiments, the ratio of viscosity of the hydrogel precursor is about 20:1. In one or more embodiments, the viscosity of the hydrogel precursor solution is from about 1 up to about 500 cP, with about 40 to about 100 cP at room temperature being particularly preferred. The pH of the alginate bath should range from about 6.2 to about 7.8 and preferably from about 6.6 to about 7.4.

The alginate shell forms from the inside out, and thickens around the droplets as the cations leach from the precursor solution droplet. In other words, the presence of the cation in the droplet causes alginate in the bath to agglomerate to the surface and crosslink around the droplet.

Next, gelation of the hydrogel precursor compound in the liquid core is initiated, such as by crosslinking and/or polymerization, to yield core/shell crosslinked microparticles. In one or more embodiments, the core/shell microparticles are combined with a hydrogel matrix crosslinker, preferably in solution. The crosslinker leaches through the alginate shell into the core/shell microparticles resulting in gelation (crosslinking) of the hydrogel precursor compound to form a 3-dimensional hydrogel matrix. The crosslinker will correspond to the hydrogel precursor compound, but can be varied to control the speed and level of crosslinking achieved within the resulting crosslinked matrix. In general, the crosslinker amount used in forming the microparticles will range from about 0.5 mM to about 30 mM depending upon the crosslinker and polymer system selected, preferably from about 1 mM to about 20 mM, more preferably from about 2 mM to about 15 mM, even more preferably from about 2.5 mM to about 10 mM, and even more preferably from about 2.5 mM to about 5 mM.

Each core/shell crosslinked microparticle comprises the alginate shell and a gelled core comprising a crosslinked, 3-dimensional hydrogel matrix. Crosslinking can be chemically induced, thermally induced, or photoinitiated, depending upon the particular precursor solution prepared. For example, the hydrogel crosslinker can be included in the alginate bath and diffuse through the alginate shell to crosslink the hydrogel microparticle in the core. Alternatively, the core/shell microparticles could be subjected to a source of UV radiation to initiate crosslinking in the hydrogel microparticle core. UV exposure times range from about 1 min. to about 10 min., preferably from about 2 min. to about 8 min., even more preferably from about 2.5 min. to about 5 min. The wavelength of the UV exposure will depend upon the photoinitiator and/or polymer system selected, but will generally range from about 100 nm to about 400 nm, preferably from about 315 nm to about 400 nm, and more preferably about 365 nm.

Suitable crosslinkers include photo- or thermal-initiated crosslinkers, chemical crosslinkers, such as acrylates, methacrylates, acrylamides, vinyl-sulfones, dithiols, and the like, which would be included as part of the alginate bath. Self-crosslinking hydrogel precursors could also be used. In these embodiments, a photoinitiator such as IRGACURE® 2959 (2-Hydroxy-4′-(2-hydroxyethoxy)-2-methylpropiophenone) or lithium phenyl-2,4,6-trimethylbenzoyl-phosphinate (LAP) may be included in the hydrogel precursor solution as the catalyst. The photoinitiator can also be included in the alginate bath. For chemical crosslinking systems, the crosslinking agent is typically provided in the alginate bath. For UV initiated crosslinking systems, the crosslinking agent can be included either in the alginate bath or in the hydrogel precursor solution along with the main polymer (backbone) species.

It will be appreciated that the precursor compounds are chemically altered in the final product via the crosslinking reaction to yield the crosslinked hydrogel matrix characterized as a porous elastic solid in particle form. For example, in one or more embodiments, the hydrogel matrix consists essentially (or event consists) of a crosslinked network of PEG and HA polymers containing thioether and ester crosslink bonds (e.g., for thiolated HA). However, it will be appreciated that the matrix may include trace amounts of unreacted precursor compounds, functional groups, and the like remaining in the network. Other examples of possible hydrogel matrix and crosslinks are described in the examples. The list provides examples, but is not exhaustive. Examples of backbone molecules that can be used include Chitosan, Agarose, Chondroitin Sulfate or a combination of molecules. Bonds formed between the backbones include chemical bonds with thioesther plus an ester, a thioesther plus an ester and a methyl on a beta carbon, a thioether sulfone, or a thioether succinimide. But other chemical bonds may be used. Photo (free radical) bonds could include ester plus ether or amide plus ether. It will be appreciated that there are increasingly complex combinations that can be utilized such that possible combinations are virtually limitless.

Once the core has been crosslinked, the alginate shell is then removed (e.g., with a chelating agent, and/or mechanical agitation, such as sonication) to yield the self-sustaining hydrogel microparticles or microbeads having the characteristics described elsewhere herein. In other words, the alginate shell is not part of the final product and is always removed before use of the microparticles. The resulting hydrogel microparticles can be collected from the solution using a mesh screen or other device, and may be rinsed or suspended in medium, as desired.

Other fabrication methods include double emulsion, spraying, and extrusion. Regardless of the fabrication method the resulting hydrogel matrix is characterized as being a semi-rigid network that is permeable to liquids and gases, but which exhibits no flow and retains its integrity in the steady state. The hydrogel microparticle is a matrix-type capsule that holds the fill material throughout the bead, rather than having a distinct shell as in a core-shell type capsule. As noted above, the hydrogel microparticle is also a self-sustaining body. In one or more embodiments, the resulting microparticles are substantially spherical in shape (it being appreciated that the spherical body does not necessarily have to be perfectly round, but may be ellipsoidal, oblong, ovoid, and the like). Advantageously, the particle size is highly customizable depending upon the capabilities of the selected droplet generator. In one or more embodiments, the resulting hydrogel microspheres or microparticles have an average (mean) maximum cross-section surface-to-surface dimension (i.e., in the case of a spherical or ellipsoidal microsphere, its diameter) of greater than 30 μm, and in some case greater than 300 μm. In one or more embodiments, the resulting hydrogel microspheres or microparticles have an average (mean) maximum surface-to-surface dimension of less than about 5 mm. Preferably, the resulting hydrogel microspheres or microparticles have an average (mean) maximum surface-to-surface dimension of less than about 2 mm, more preferably from about 30 μ to about 2 mm, even more preferably ranging from about 50 μ to about 1.5 mm, more preferably from about 150 μm to about 1.5 mm, even more preferably from about 300 μm to about 1.4 mm. In some cases, smaller microparticles ranging from about 30 to about 750 μm or 500 μm in size can be formed. In certain indications, it may be beneficial to produce a diverse range of sizes for a single application. In other situations, it may be beneficial for a product to have a more uniform range of microparticle diameters. For ease of reference, this cross-section dimension is referred to herein simply as the “size” of the microparticle. In any event, the hydrogel microparticles of the invention are not nanosized and would not be considered nanoparticles or any other kind of nanocrystalline shape.

The durability of the microparticles can be adjusted by changing various parameters of the microparticle, including in the formulation and/or the processing parameters, such as polymer precursor mass fraction or molecular weight, crosslinker molecular weight, ratio of crosslinker and polymer precursor, crosslinker hydrolysis, crosslinking kinetics, crosslinking time, e.g., UV exposure time, and combinations thereof. Additional hydrogels are described in co-pending PCT/US2020/036361, filed Jun. 5, 2020, and incorporated by reference herein in its entirety.

The present microparticles are not for delivery of small molecule therapeutics or cells or tissues, and are “empty” —that is, substantially free of such drugs, biologics, cells, tissues, or other therapeutic payload encapsulated therein or attached thereto. The microparticles are also substantially free of metals or plastics. The term “substantially free,” as used herein, means that the ingredient is not intentionally added to the composition, although incidental impurities may occur, or residual/trace amounts may be left behind from the manufacturing process. In such embodiments, the hydrogel precursor solution compositions comprise less than about 0.05% by weight, preferably less than about 0.01%, and more preferably about 0% by weight of such an ingredient, based upon the total weight of the solution taken as 100% by weight. The only payload contemplated herein is non-therapeutic in nature, such as a dye or other detectable label that can be used to visualize the microparticles.

Also described herein are compositions that comprise a plurality of 3-dimensional hydrogel microparticles suspended in a pharmaceutically-acceptable delivery vehicle. The resulting viscosupplement will comprise from about 0.1% to about 60% wt/wt, preferably from about 3% to about 60% wt/wt of microparticles based upon the total weight/volume of the viscosupplement taken as 100% by weight. The vehicle is preferably selected to be suitable for localized delivery (direct injection) at the site of implantation, such as through a small gauge needle. In one or more embodiments, the hydrogel microparticles may be lyophilized and stored as a dry powder before being reconstituted with suitable aqueous vehicle. In one or more embodiments, the hydrogel microparticles can be cryopreserved for storage and do not exhibit decrease in matrix quality upon thaw.

In one or more embodiments, methods of viscosupplementation are contemplated herein. The method generally comprises injecting or implanting a plurality of hydrogel microparticles into or near a site of implantation in a subject. The implanted hydrogel microparticles provide cushioning and/or reduce friction or wear between tissues at the site of implantation. As demonstrated in the working examples, the microparticles also induce a favorable physiological response in the subject at the site of implantation. For example, the microparticles do not induce an inflammatory response at the site of implantation, while encouraging an increase in healing, such as by an increase in cartilage production and/or the quality (smoothness) of the cartilage produced. In one or more embodiments, methods contemplated here involve reducing pain and/or providing viscosupplementation in a joint by introducing into the joint a plurality of the microparticles. The microparticles help maintain a structure and cushion between cartilage surfaces to prevent cartilage contact. Moreover, due to their viscoelastic nature, they demonstrate an elastic response during articulation and load bearing to absorb load and prevent cartilage contact, while still allowing the cartilage surfaces to move across one another with low friction (owing to the smooth outer surface of the microparticle bodies). This, in turn, further reduces pain, inflammation, and further damage at the site of implantation.

The methods can be applied in a variety of human or animal joints, including, without limitation, knees, hips, ankles, wrists, elbows, shoulders, toes, fingers, and spine. Preferably, the microparticles or composition comprising the microparticles is introduced into an intraarticular space of the joint, more preferably into the synovial capsule portion of the joint. The joint may be one affected with OA or at risk of developing OA. The methods also include using the microparticles as a surgical lubricant, wherein the microparticles are applied in and/or around a site of implantation of a surgical device, such as a surgical mesh, etc. or between organs, to reduce friction and irritation with surrounding tissue. The methods also include use of the microparticles as dermal fillers as well as in wound healing.

For treatment methods, the microparticles are suspended or dispersed in a suitable delivery vehicle for administration to the subject. Exemplary delivery vehicles will include biocompatible liquid suspensions, viscous solutions, putties, pastes, or gels in which the microparticles are distributed, such as synthetic synovial fluid, and uncrosslinked/low concentration HA liquid solutions. Saline solutions or other buffered solutions may also be used as vehicles for delivery. More generally, the methods comprise (or consist of) locally administering a therapeutically effective amount to treat the location of inflammation, injury, arthritis, surgery, degeneration, etc. in the patient. Dosages will differ for various joint or surgical applications and the size/species of the patient to be treated. Example injection dosages for treatment of the human knee, shoulder, spine, or sacroiliac joint include, include but are not limited to, about 0.25 mL to about 10 mL, and more preferably from about 1 mL to about 4 mL per joint. For the human hip, the volume range could be between about 0.5 mL to about 15 mL, or more preferably from about 1 mL to about 6 mL per joint. Application to the human hand or other smaller joints such as the feet and toes would require smaller doses in the range of about 0.01 mL to about 2 mL per joint. In the veterinary application, horse joints would require larger volumes from about 0.5 mL to about 15 mL per joint for the fetlock, knee, or stifle. Dogs and cats would require smaller doses of approximately 0.10 mL to about 5 mL for joint applications. While the injections are intended to decrease the frequency of administrations to the patient, it is expected that repeated treatments to the same or different joints may be necessary.

The injected composition could contain a single formulation of microparticles within a narrow size range, for example 700-1000 mm diameters. Conversely, the composition could be a mixture of formulations having different microparticle sizes. For example, it may be advantageous to mix different formulations of hydrogels to have a combination of microparticles that degrade at different time points, or have different stiffness characteristics. Thus, composition of heterogenous microparticles are contemplated herein, comprising a first population of microparticles having a first characteristic (e.g., size range, stiffness, and/or degradation profile, etc.) and a second population of microparticles having a second characteristic (e.g., size range, stiffness, and/or degradation profile, etc.), where the second characteristic is different from the first characteristic. The heterogenous compositions can include 2, 3, 4, 5, 6, 7, 8, 9, or 10 different microparticle formulations or more. Likewise, the viscoelastic microparticles of the invention may be mixed with viscous HA that is not crosslinked or only partially crosslinked to or other viscous fluid joint supplement (as the carrier vehicle) yield a suitable therapeutic formulation. The product could, for example, be composed of a 1:10 ratio of microparticles to a viscous fluid joint supplement such as partially crosslinked HA.

Administration generally includes direct injection of the microparticle composition at or near the site of inflammation, injury, arthritis, surgery, degeneration, etc., such as into the joint or between tissues. Advantageously, the durable microparticles are generally confined to the localized region for a therapeutically effective period of time. As used herein, the term “therapeutically effective” refers to the amount and/or time period that will elicit the biological or medical response of a tissue, system, animal or human that is being sought by a researcher or clinician, and in particular elicit some desired therapeutic effect. For example, in one or more embodiments, therapeutically effective amounts and time periods are those that reduce inflammation and initiate or promote healing of the site of inflammation, injury, arthritis, degeneration, etc. One of skill in the art recognizes that an amount or time period may be considered therapeutically effective even if the condition is not totally eradicated but improved partially.

The treatment may be repeated via additional injections or infusions if necessary. Those skilled in the art will appreciate that treatment protocols can be varied depending upon the particular inflammation, injury, arthritis, surgery, degeneration, condition or healing status, and preference of the medical or veterinary practitioner or researcher.

Advantageously, the hydrogel microparticles are durable, meaning they will not break down or degrade under storage conditions for at least 6 months in Phosphate Buffered Saline (PBS) at 37° C. In other words, the durable microparticles have “in vitro storage stability” of greater than 6 months. In one or more embodiment, durable hydrogel particles are shelf-stable in PBS at room temperature (27° C.) for one year or more, with current data exemplifying microparticles with a shelf-life of more than 4 years. Preferably, when such durable hydrogel microparticles are implanted, they will not break down for at least 3 months, and more preferably for at least 6 months under normal physiological (aka normal in vivo clearance of foreign bodies via phagocytosis, degradation, adsorption, etc.) conditions. Thus, in one or more embodiments, benefits of viscosupplementation using the hydrogel microparticles can be achieved with a single injection, which remains effective for an extended period of time. That is, treatment regimens using these inventive viscosupplements do not require multiple injections for a single treatment regimen, nor do they require administering the hydrogel microparticles in weekly or monthly intervals as with existing treatments. In one or more embodiments, the hydrogel microparticles are administered as a single injection, which does not have to be repeated for at least 6 weeks, and in some cases at least 10 weeks or longer.

A particular advantage of the inventive compositions and microparticles is their improved “stickiness” as compared to other types of microparticles when implanted. That is, the microparticles have a tendency to adhere or cling to the tissue in vivo at the site of implantation and shown in the photographs in the examples below. This further enhances the effectiveness of the treatment by maintaining the localized lubrication at the site of implantation. Further, the microparticles demonstrate no induction of any inflammatory response at the site of implantation, and/or any foreign body response, including lymphocytes or collagen ring formation.

Table A below provides a comparison of current commercially available HA-based joint lubricants and compares them to two versions of the inventive microparticles: one manufactured from HA and another from polyethylene glycol (PEG). The table illustrates the significantly higher molecular weight of the final products along with high crosslinking, and greatly expanded stiffness. Stiffness directly correlates to the strength of the crosslinkers which relates to the duration of the hydrogel in the body. The stiffness, and thus duration, is drastically greater in the inventive microparticles.

Surgical procedures often require the placement of lubricants at the internal site of the surgical intervention to avoid formation of fibrotic scarring in the area. This is particularly important when devices are inserted into the body with the intention of remaining there long term, such as in the use of mesh for hernia repair or placement of a cardiac pacer. The application of a lubricant at the time of surgery can often inhibit scar formation and improve the recovery.

In addition to the numerous human applications for the crosslinked microparticles in the treatment of human joint diseases and trauma, there is broad application for the product in the veterinary practice. This includes lameness in horses and joint dysplasia in dogs and cats. The Table B below summarizes a comparison of the characteristics of the inventive microparticles to products sold in the veterinary market.

TABLE A
Human

| | Precursor | Matrix | Crosslinking | Viscosity | Number of | HA/PEG conc | Stiffness |
|---|---|---|---|---|---|---|---|
| | MW (MDa) | MW (MDa) | (%) | (Pa) | injections | (mg/mL) | (Pa) |
| HA | 0.20 | Indeterminate | 90-100 | Solid | 1 | 40 | 20,000 (E′) |
| Microparticles | | due to complete | | microbeads | | | |
| | | crosslinking | | | | | |
| PEG | 0.0034 | Indeterminate | 90-100 | Solid | 1 | 18 | 726,000 (E′) |
| Microparticles | | due to complete | | microbeads | | | |
| | | crosslinking | | | | | |
| Hyalgan (HA) | 0.5-0.7 | 0.5-0.7 | No | NR | 3-5 | 10 | 0.6 |
| Synvisc (HA) | 6.0 | 6.0 | Yes, partially | 25 | 3 | 8 | 111 (G′) |
| Orthovisc (HA) | 1.0-2.9 | 1.0-2.9 | No | 46 | 3-4 | 15 | 60 |
| Supartz (HA) | 0.6-1.2 | 0.6-1.2 | No | 20 | 3-5 | 10 | 12 |
| Gel-One (HA) | NR | NR | Yes, partially | NR | 1 | 10 | NR |
| Euflexxa (HA) | 2.4-3.6 | 2.4-3.6 | No | NR | 3 | 10 | NR |
| Durolane (HA) | 6.0 | 6.0 | Yes, lightly | 60-70 | 1 | 20 | 500-600 |
| Monovisco (HA) | 1.0-2.9 | 1.0-2.9 | Yes, lightly | NR | 1 | 22 | NR |

NR = not reported

TABLE B
Veterinary

| | Precursor | Matrix | Crosslinking | Viscosity | | HA/PEG conc |
|---|---|---|---|---|---|---|
| | MW (MDa) | MW (MDa) | (%) | (Pa) | Administration* | (mg/mL) |
| HA | 0.2 | Indeterminate | 90-100 | Solid | 1 injection, stays | 40 |
| Microparticles | | due to complete | | microbeads | in joint 6 weeks | |
| | | crosslinking | | | | |
| PEG | 0.0034 | Indeterminate | 90-100 | Solid | 1 injection, stays | 18 |
| Microparticles | | due to complete | | microbeads | in joint ~10 weeks | |
| | | crosslinking | | | | |
| Noltrex | 10 | 10 | Minimally (less | NR | 1-2 injections, | 4 |
| (Polyacrylamide) | | | than 2.5%) | | Stays in joint up | |
| | | | | | to 60 days | |
| Legend (HA) | NR | NR | None | NR | 3 injections over | 10 |
| | | | | | 3 week period. | |
| | | | | | Stays in joint | |
| | | | | | for <24 hours | |
| Arthamid/ | NR | NR | Minimally (less | NR | 1-2 injections | 2.5 |
| Synamid | | | than 2.5% | | 4 weeks apart | |
| (Polyacrylamide) | | | | | | |

NR = Not Reported
*Dosing information from manufacturer

The characteristics reported in the tables above demonstrate that the inventive microparticles have higher crosslinking and significantly greater stiffness for longer duration compared to currently available products.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Characteristics of the Final Hydrogel Microparticles

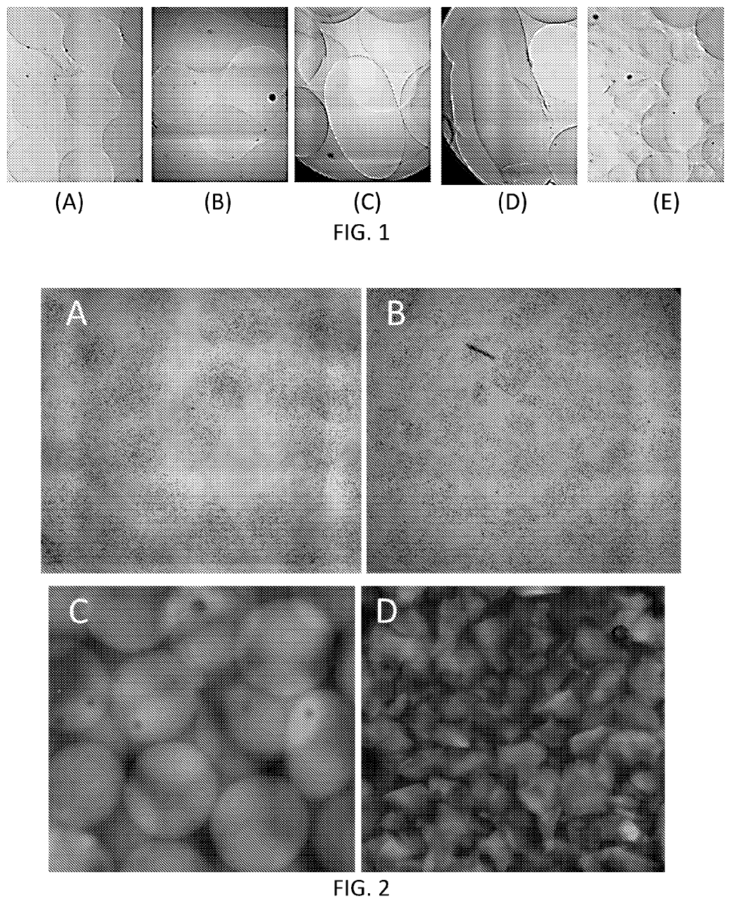

Crosslinked microparticles can be constructed using a number of different manufacturing methods including (but not limited to): core shell spherification, emulsion, extrusion of alginate, patterned molds, and printed. Microparticles will typically be small enough to be injected through a needle or infused through a catheter. We have manufactured microparticles in the size ranges of under 400 μm to over 1,500 μm, so that they can pass through 18 to 25 G needles, which makes them easily injected into the joint space or other body cavities. The size and shape of the microparticles can be altered to meet the therapeutic goal. FIG. 1 illustrates examples of microparticles as spheres (FIG. 1A), teardrops (FIG. 1B), ovals (FIG. 1C), and organic large tube shapes (FIG. 1D). The product could also be shaped as tubes, rectangles or other shapes. In addition, a mixture of shapes could be manufactured and used in the same product as shown in FIG. 1E. The particles can withstand physical forces such as shearing. However, when they are put under excessive force they break up into smaller pieces but maintain a distinct physical shape. This is different from paste-like lubricant gels, which do not maintain a physical border and distinct shape. FIG. 2 shows MeHA microparticles prior to exposure to shear forces (FIG. 2A) and after (FIG. 2B). AHA microparticles impregnated with a fluorescent probe are shown prior to exposure to shear forces (FIG. 2C) and after (FIG. 2D).

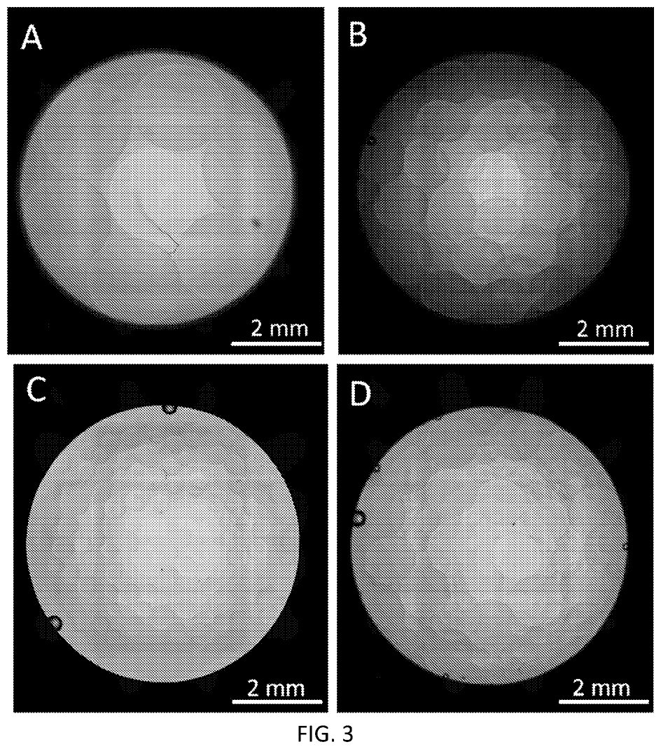

Likewise, the microparticles can be created in a wide range of sizes from an average of 200-2000 microns as shown in FIG. 3. Large microparticles are shown in FIG. 3A, medium size in FIG. 3B, small microparticles in FIG. 3C and a mixture of sizes in FIG. 3D.

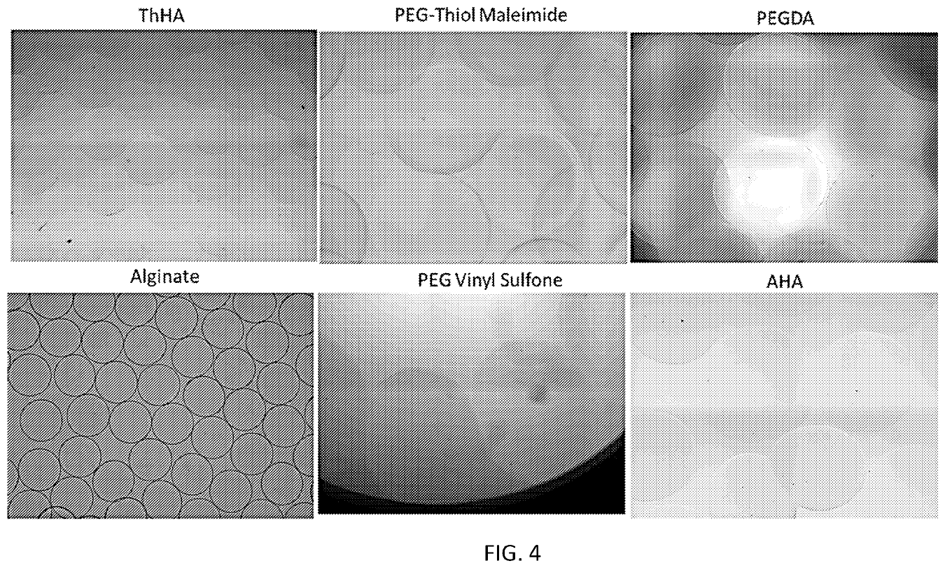

By utilizing different precursor polymers and crosslinking schemes the crosslinked microparticles can be manufactured from a variety of materials. FIG. 4 provides a few examples of microparticles made from thiolated hyaluronic acid (ThHA), polyethylene glycol dithiol maleimide, (PEG-Thiol Mal), diacrylate polyethylene glycol polyethylene diacrylate (PEGDA), alginate, polyethylene vinyl sulfone, and acrylated hyaluronic acid (AHA).

The hydrogel microparticles can advantageously be fabricated from a variety of slow-gelling polymer precursors, such as hyaluronic acid (HA) and polyethylene glycol diacrylate (PEGDA), polyethylene glycol maleimide (PEGMAL), multi-arm PEGs, hyaluronan, fibrin, chitosan, collagen, heparin, polylactic acid (PLA), poly(L-lactic acid) (PLLA), polylactic-co-glycolic acid (PLGA), polycaprolactone (PCL), polyvinyl alcohol (PVA), polyvinylpyrrolidone (PVP), methacrylic acid (MAA), 2-hydroxyethyl methacrylate (HEMA), polyacrylamide (PAM) extracellular matrix, and functionalized varieties of the same (e.g., acrylated, methacrylated, thiolated, etc.) or multi-arm varieties of the same (e.g., 4-arm PEG, 8-arm PEG). However, any crosslinkable hydrogel precursor compounds would be suitable for use with the invention, with preferred compounds being biocompatible homopolymers or copolymers, and particularly block copolymers, as well as other types of crosslinkable monomers and/or oligomers.

Further characterization of some of the chemical version of the end products are provided in Table 1. The first row indicates the variety in precursor characteristics including the precursor chemistry, the mass fraction and viscosity (ranging from 50 to 190cST) prior to crosslinking. Different crosslinking mechanisms can be utilized including photo-initiation and chemical reactions (Table 1) and as such the different crosslinking approaches require different amounts of time to cure as shown in Table 1.

TABLE 1

| Attribute | AHA | | ThHA | | MeHA | | PEGDA | |
|---|---|---|---|---|---|---|---|---|
| Precursor polymer mass | 4% AHA 200 kDa | | 1.2% ThHA | | 2.5% MeHA | | 18% PEGDA 3.4 kDa | |
| fraction | | | | | 1% PEGDA 3.4 KDa | | 12% PEGDA 20 kDa | |
| Precursor viscosity | 50 | cST | 90 | cST | 190 | cST | 60 | cSt |
| Crosslinking mechanism | Photo initiated: free | | Chemical: thiol-acrylate | | Photo initiated: free | | Photo initiated: free radical | |
| | radical polymerization | | | | radical polymerization | | polymerization | |
| Crosslinking Time | 120 | sec | 35 | min | 60 | sec | 60 | sec |
| Product polymer mass | 0.39% | | 3.6% | | 0.95% | | 3.5% | |
| fraction | | | | | | | | |
| Mass Swelling Ratio | 256.0 | | 27.7 | | 105.7 | | 28.5 | |
| “Q”* | | | | | | | | |
| Average Microparticle | 1095 | mm | 637 | mm | 1156 | mm | 904 ± 142 | um |
| diameter (final swollen) | | | | | | | | |
| Young's elastic modulus | 0.05-50 | kPa | 0.05 kPa-1 MPa | | 0.05 kPa-100 kPa | | 1 kPa-2.5 MPa | |
| Microparticle diameter | 250-2500 | mm | 400-1000 | mm | 700-1500 | mm | 100-2000 | mm |

range

*Swelling ratio “Q” is the ratio of the hydrated equilibrium mass to the dry mass of the gels.

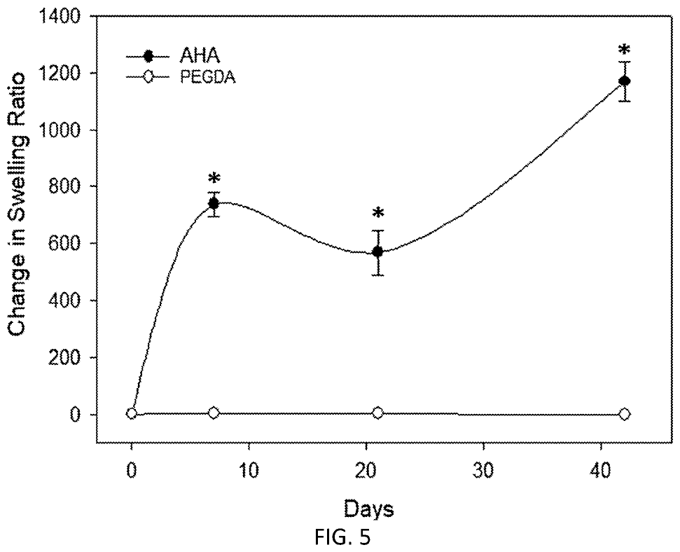

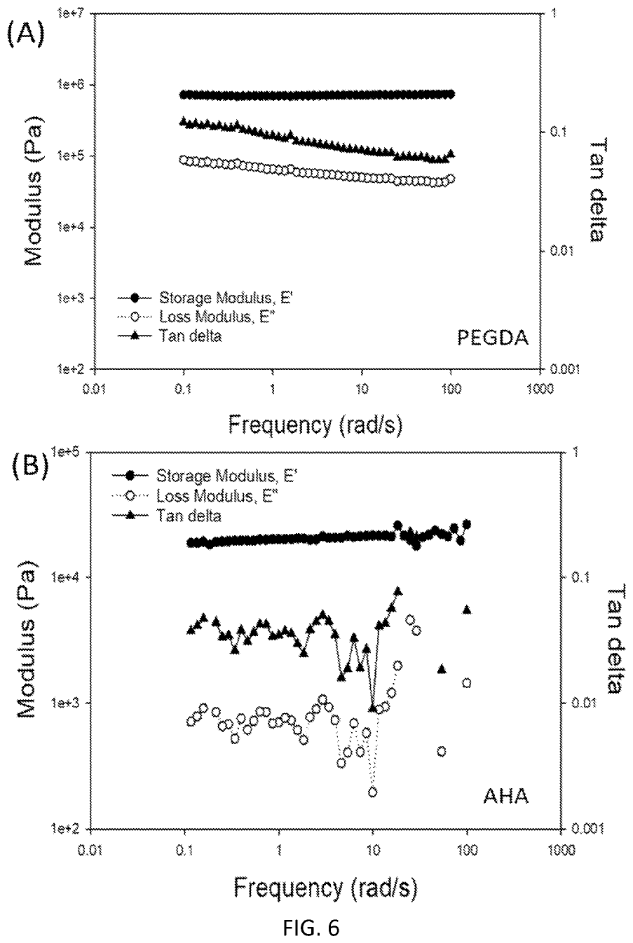

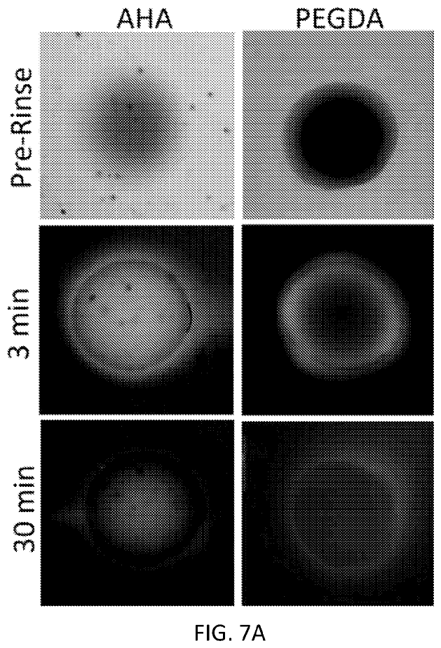

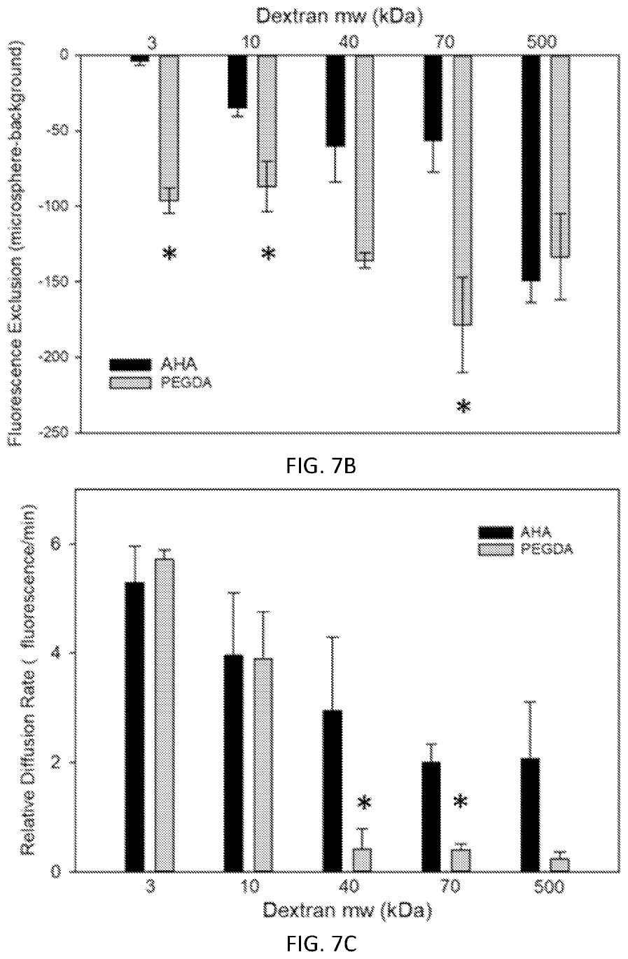

The final microparticles can have vastly different mass fractions, ranging from 0.39 to 3.6% and great variation in the subsequent Q values (swelling ratios) ranging from 28 to 256 (Table 1). The average diameters of the particle sizes and the range of sizes also varied from 397 to 1156 and was related to the swelling ratio. The swelling ratios were found to change over time for some of the hydrogels. For example, AHA showed a dramatic increase in the swelling ratio initially and then continued to rise over the 42 days it was tracked (FIG. 5). In contrast the PEGDA microparticles did not change in the swelling ratio over the same period of time. Associated with the differences in Q values are the differences in the rheological characteristics of different hydrogels shown in FIG. 6. As the figure demonstrates the E′ was greater than the E″ for both hydrogels, indicating that they are solid-like in composition. The diffusion characteristics of hydrogel microparticles are also related to the swelling and rheological values. For example, AHA microparticles are more diffuse with a higher Q value, compared to PEGDA microparticles (FIG. 7).

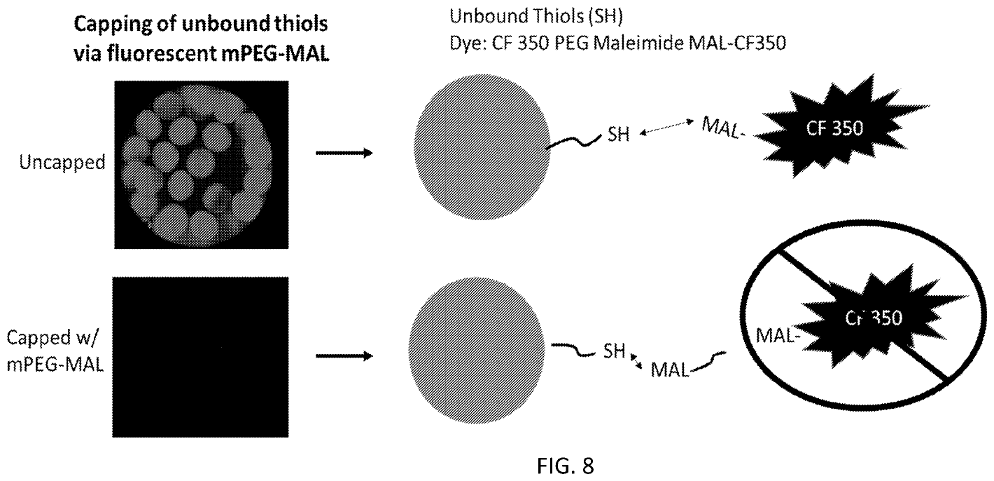

In addition to different crosslinking approaches, additional chemical modifications can be made to alter the characteristics of the microparticles. For example, unreacted species within the fully-crosslinked particle can be further modified as shown in FIG. 8. This can be done to alter the interaction of the particle with the surrounding tissue either making it less reactive or more reactive, depending on the application. In this example, low levels of residual reactive groups are commonly present within a hydrogel after crosslinking. These groups can be utilized, if desired, for downstream functionalization. In this example, a monofunctional PEG-maleimide “molecular cap” is used to quench residual thiol groups. As seen in the example, treatment of the hydrogel spheres with the molecular cap successfully blocked binding of a maleimide conjugated fluorescent dye, in contrast to untreated spheres which readily bound the dye.

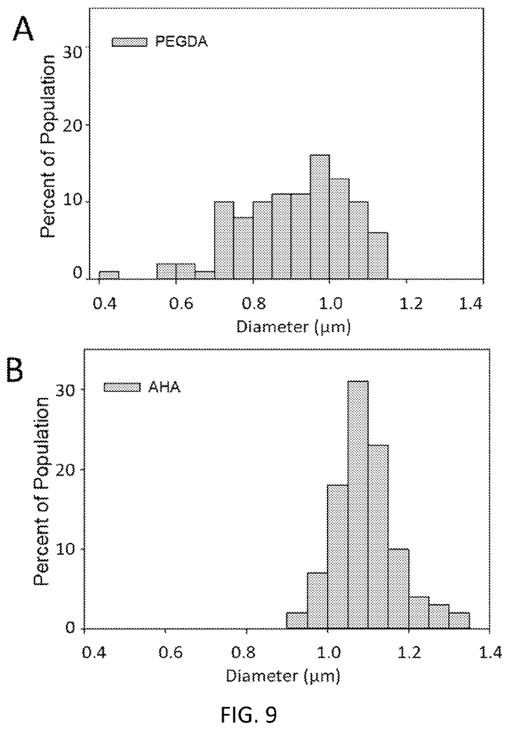

Single batches of microparticles can have sizes that are within a narrow range of diameters or have a wider range, which can be designed for the given application. FIG. 9 illustrates the difference in batch monodispersity from two different hydrogel formulations.

### In Vitro Degradation Rate

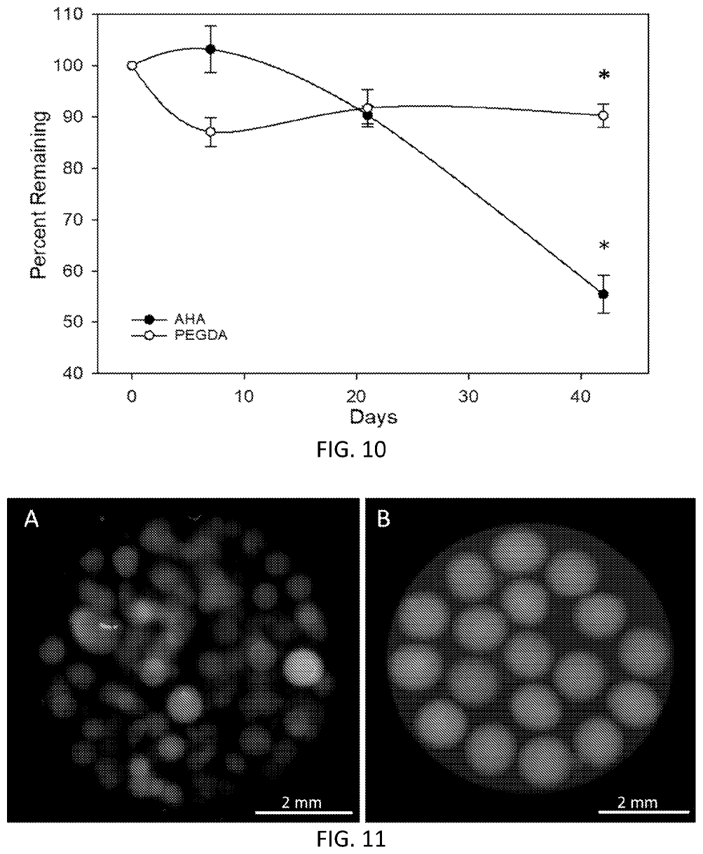

In contrast to these uncrosslinked forms of HA, our fully-crosslinked HA microparticles have an in vitro half-life of approximately 50 days while microparticles made of polyethylene glycol have an indefinite duration in vitro (FIG. 10). In this experiment microparticles were maintained at 37° C. and 10% humidity for the 50 days.

In the body, the degradation of the microparticles is dependent on endogenous enzymes such as hyaluronidase. In vitro studies in the preliminary stages will demonstrate the enzymatic degradation of the fully-crosslinked microparticles. To be able to monitor the microparticle degradation in vivo, we developed fluorescent microparticles. SAMSA Fluorescein was added to the precursor AHA solution shown in FIG. 11A. FIG. 11B shows fluorescent labeling of PEGDA microparticles.

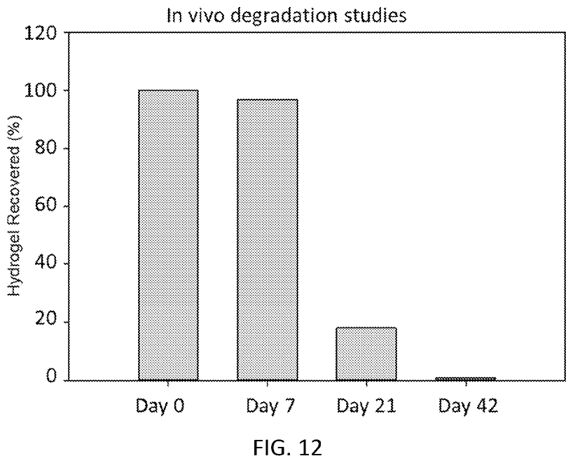

The in vivo degradation rate of the microparticles with a higher Q values (the AHA microparticles) were monitored in weight bearing rat knees. At selected time points, the knees were analyzed for the remaining microparticles. As shown, this formulation degraded in vivo in approximately 6 weeks (FIG. 12). However, the in vivo degradation time can increased or decreased as desired by appropriate chemical modifications such as using different crosslinkers, the density of crosslinking, the length of the crosslinkers, and concentration of the base polymer.

### Biocompatibility of Microparticles

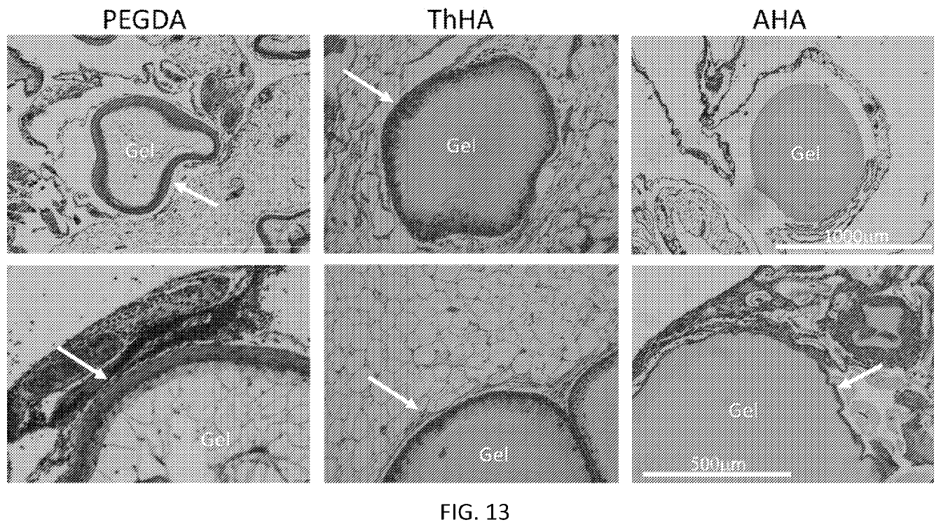

The biocompatibility of microparticles is exemplified in FIG. 13 where hydrogel particles made of PEGDA, ThHA, or AHA, using the same technology. The microparticles were injected intraperitoneally into rats and after 2 weeks, intact hydrogel microspheres were still found in the region. Areas of fibrosis (collagen ring) and inflammatory response (macrophages and lymphocytes) were measured at 4 different locations around each microsphere by 2 blinded technicians. Only limited fibrosis was found surrounding some of the microparticles (FIG. 13). In fact, only 28.8% of the AHA microbeads showed any signs of a surrounding fibrotic area while far more of the PEGDA beads had some fibrosis (Table 2). When fibrosis was found surrounding the AHA microparticles, it was much smaller than particles made of other components. In addition, we measured the width of fields of inflammatory cells surrounding the microparticles. Fewer samples showed signs of inflammatory cells surrounding the microparticles with 71% of the samples form PEGDA and 16% for the AHA microparticles and the average width of those regions was minor (Table 2).

| Foreign Body Response | PEGDA | AHA |
|---|---|---|
| % of spheres with fibrosis | 92.4% | 28.8% |
| Width of fibrosis when present | 72.28 +/− 37.82 | 30.63 +/− 32.83 |
| % of spheres with lymphocytic | 70.5% | 16.3% |
| response | | |
| Width of lymphocytic region when | 56.93 +/− 83.52 | 26.01 +/− 76.81 |
| present | | |

### Protection of the Osteoarthritis Joint

To test the effectiveness of the microparticles in managing osteoarthritis, arthritic rats were injected with small AHA microparticles. We tested two different rat osteoarthritic models, the destabilization of the medial meniscus (DMM) and the articular cartilage defect model (CD).

For the CD model, 9-12 weeks old male Sprague-Dawley rats received direct manual damage to the articular cartilage under sterile conditions. The knee joint was exposed through a medial parapatellar incision and the joint capsule incised. The patella was displaced laterally and the patellar tendon kept intact and carefully protected during the procedure. A 2.0×4.0 mm articular cartilage defect (CD) was created in the patellar groove of the femur. Tissue debris was rinsed out with normal saline. The crucial ligaments and other ligaments around the knee joint were preserved to maintain the stability and mobility of the joint after surgery. The patella and patellar tendon were reduced to their original position after creating the articular cartilage defect. The joint capsule was sutured closed to assure proper use of the knee after surgery.

For the DMM model, 9-12 week old Sprague Dawley rats were anesthetized prior to the surgical procedure performed on the knee joints under a dissecting microscope. The knee joint was exposed through a medial parapatellar incision and the joint capsule was incised. The patella and the patellar tendon were kept intact and carefully protected during the procedure. The medial meniscotibial ligament (MML) anchors the medial meniscus to the tibial plateau. After careful exposure of the MML, it was transected with surgical scissors to destabilize the medial meniscus which induces post-traumatic osteoarthritis. The crucial ligaments and other ligaments around the knee joint were preserved to maintain the stability and mobility of the joint after surgery. The joint capsule was closed and skin sutured closed.

At four weeks post-surgery, hydrogels were injected into the right knee and vehicle (PBS alone) into the left knee (control) for both animal models (DMM and CD). Animals were euthanized at either 6 or 12 weeks post-injection of the microbeads and the knee joints harvested and processed for histological and other laboratory analyses.

Examples of images collected show improvement with the microbead injection. FIG. 14 provides examples of rat knees following the CD model of osteoarthritis 12 weeks after the injection of the microbeads or vehicle. The full knee sections are shown in FIG. 14 with clear indication of the site of the removed cartilage, the indented area identified by the red arrows. In the knees that received an injection of the vehicle control (FIG. 14A) the articular surface is jagged and there is no staining (red) for cartilage at the joint surface. The deep red indicated by the black arrow represents the growth plate, which is not affected by the treatment. In rats that received microparticles (FIG. 14B), there was red staining at the joint surface indicating remaining cartilage at the surface of the joint. In addition, the articular surface was smoother with no jagged edges, compared to the vehicle (white arrows).

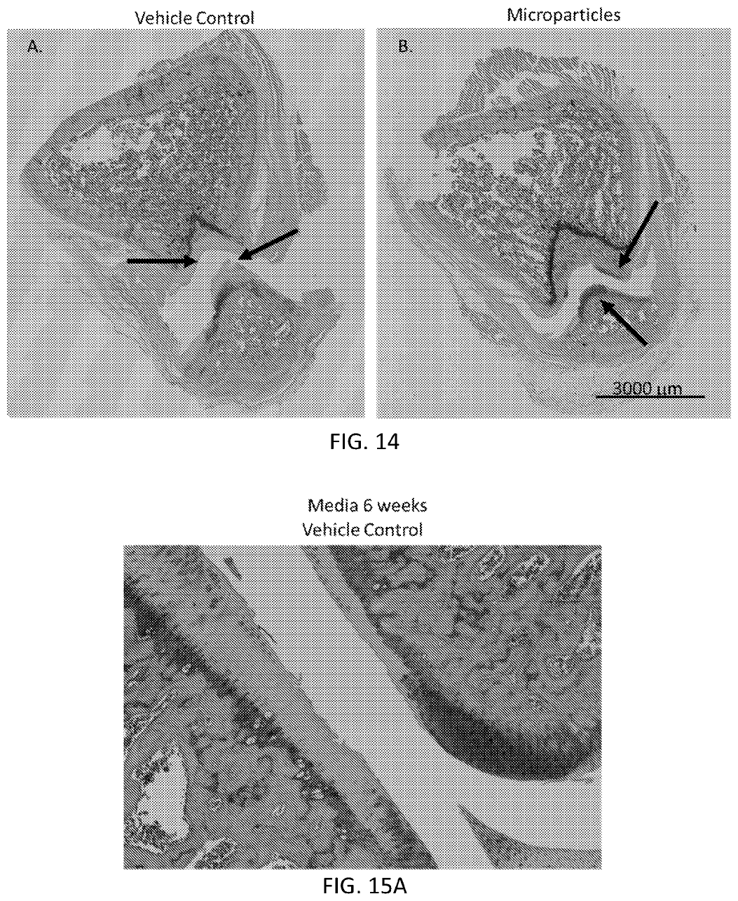

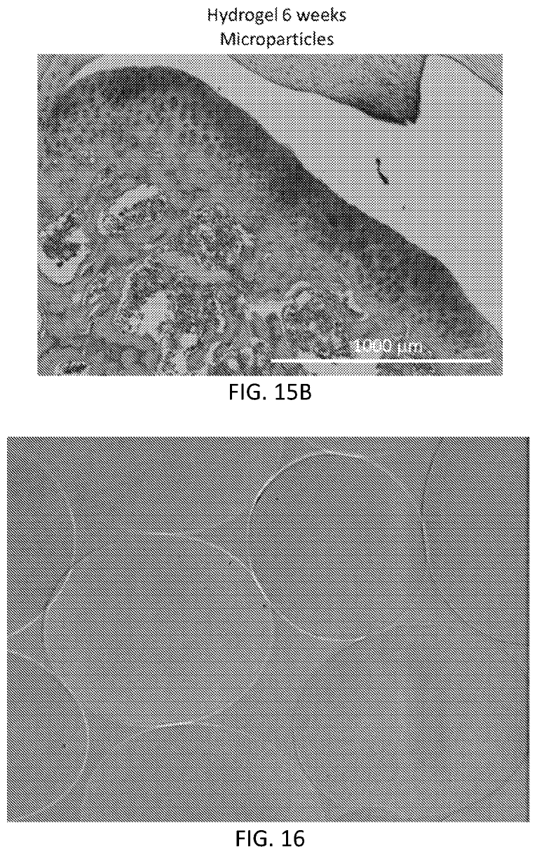

Higher magnification images of the DMM rat osteoarthritis model are shown in FIG. 15. Six weeks after the injection of vehicle or microbeads, the animals were euthanized, and tissue stained for cartilage (red). At the higher magnification the rough edges of the joint along with the lack of cartilage are obvious in the vehicle-injected controls (FIG. 15A). Alternatively, the knee that received the microbeads had more cartilage tissue (red stain) and a smoother edge (FIG. 15B).

### Shelf Life of Microbeads

Informally, we have maintained PEG-based hydrogel microbeads at room temperature in PBS for over 4 years with no visible deterioration of the physical properties. FIG. 16 provides 2 example images of thiolated hyaluronic acid stored at room temperature in water for more than 4 years and then imaged. More formally, we have cryopreserved microspheres and determined their structural integrity following thawing.

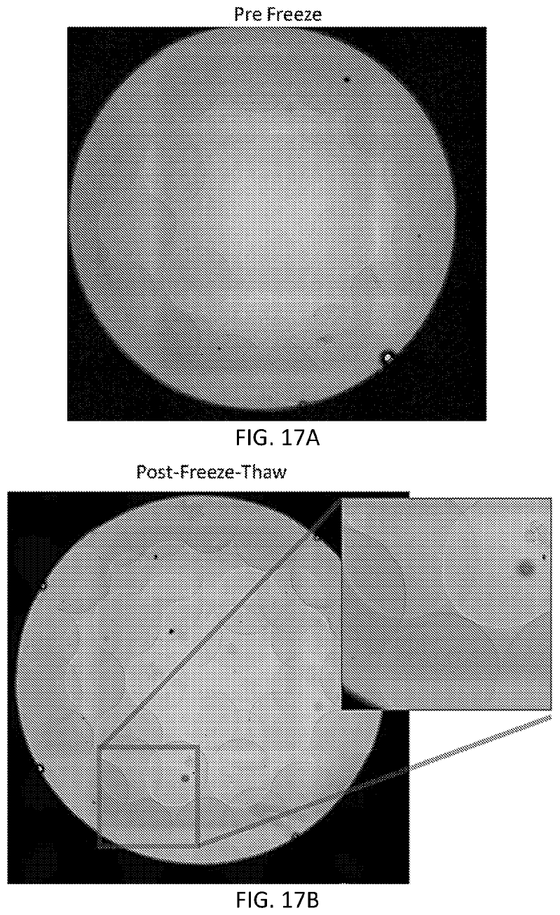

The final crosslinked product can be frozen without negatively affecting the chemistry of the physical properties of the microparticles using a slow-freeze protocol and found that upon thawing by placing at 37C for approximately 2 minutes, the microparticles maintain their structure and surface topography. FIG. 17 illustrates pictures of the microparticles before cryopreservation 25 at −80° C. and after cryopreservation and thawing with no change in size, shape or microscopic structure.

1. A method for viscosupplementation in a joint or surgical site of a human or animal subject, said method comprising: introducing into the joint or surgical site a plurality of porous viscoelastic solid hydrogel microparticles having a size of greater than about 30 μm, and substantially free of any cells, tissue, or therapeutic compounds.

2. The method of claim 1, wherein said hydrogel microparticles are injected at or near the joint or surgical site.

3. The method of any one of the foregoing claims, wherein said hydrogel microparticles are characterized as self-sustaining bodies having a 3-dimensional matrix of covalently or ionically crosslinked polymer compounds.

4. The method of any one of the foregoing claims, wherein said plurality of porous viscoelastic solid hydrogel microparticles comprise at least two different populations of microparticles, wherein a first population of microparticles comprises a plurality of microparticles having a first characteristic, and a second population of microparticles comprises a plurality of microparticles having a second characteristic, wherein said first characteristic is different from said second characteristic.

5. The method of any one of the foregoing claims, wherein said hydrogel microparticles provide cushioning and/or reduce friction between tissues at or near the joint or surgical site.

6. The method of any one of the foregoing claims, wherein said hydrogel microparticles induce a favorable physiological response in the subject at the site of implantation.

7. The method of any one of the foregoing claims, wherein said hydrogel microparticles do not induce inflammation.

8. The method of any one of the foregoing claims, wherein said hydrogel microparticles increase amount or quality of cartilage produced in a joint at the site of implantation.

9. The method of any one of the foregoing claims, wherein said hydrogel microparticles provide a resilient response to load-bearing for cushion and reduce friction within a joint at the site of implantation.

10. The method of any one of the foregoing claims, wherein said hydrogel microparticles are introduced into a joint of the knees, hips, ankles, wrists, elbows, shoulders, toes, fingers, or spine.

11. The method of any one of the foregoing claims, wherein said hydrogel microparticles are injected into an intraarticular space of a joint.

12. The method of any one of the foregoing claims, wherein said hydrogel microparticles are introduced into a synovial capsule portion of said joint.

13. The method of any one of the foregoing claims, wherein said hydrogel microparticles are introduced in and/or around a site of implantation of a surgical device in said subject.

14. The method of claim 13, wherein said surgical device is surgical mesh.

15. The method of any one of the foregoing claims, wherein said hydrogel microparticles are introduced subdermally as a dermal filler.

16. The method of any one of the foregoing claims, wherein said hydrogel microparticles are introduced between organs of said subject.

17. The method of any one of the foregoing claims, wherein said hydrogel microparticles are suspended or dispersed in a suitable delivery vehicle.

18. The method of claim 16, wherein said delivery vehicle is a buffered saline solution, viscous solution, putty, paste, or gel.

19. The method of any one of the foregoing claims, wherein said hydrogel microparticles are directly injected into the site of implantation.

20. The method of any one of the foregoing claims, wherein said hydrogel microparticles are provided in a composition in a pre-loaded syringe for injecting into the joint or surgical site.

21. The method of any one of the foregoing claims, wherein said hydrogel microparticles are confined to said site of implantation for a therapeutically effective period of time.

22. The method of any one of the foregoing claims, wherein said hydrogel microparticles remain at said site of implantation without degradation under normal physiological conditions for at least 3 months.

23. The method of any one of the foregoing claims, wherein said hydrogel microparticles remain at said site of implantation without degradation under normal physiological conditions for at least 6 months.

24. The method of any one of the foregoing claims, wherein said human or animal subject received recurring dosages of said hydrogel microparticles administered at an interval of every 3 months, preferably every 3-6 months, preferably every 6-12 months.

25. A kit for viscosupplementation in a joint or surgical site of a human or animal subject, comprising a plurality of porous viscoelastic solid hydrogel microparticles having a size of greater than about 30 μm, and substantially free of any cells, tissue, or therapeutic compounds, and instructions for administering the same.

26. The kit of claim 25, wherein said plurality of porous viscoelastic solid hydrogel microparticles are lyophilized, said kit further comprising a biologically compatible vehicle in a separate container for reconstituting said hydrogel microparticles or instructions for reconstituting said hydrogel microparticles prior to administration.

27. The kit of claim 26, further comprising an empty syringe and instructions for loading said syringe with said hydrogel microparticles after reconstitution.

28. The kit of claim 25, wherein said plurality of porous viscoelastic solid hydrogel microparticles are packaged in solution, wherein said solution is pre-loaded into a syringe for administration and instructions for the same.

29. The kit of claim 25, wherein said plurality of porous viscoelastic solid hydrogel microparticles are packaged in solution wherein said kit comprises an empty syringe and said hydrogel microparticles in a separate container configured to permit withdrawal of the solution from the container to load said syringe prior to said administration, and instructions for the same.

30. A viscosupplement for use in treating a joint or surgical site of a human or animal subject such as by lubricating and cushioning intra-articular joint surfaces and tissues, comprising a plurality of porous viscoelastic solid hydrogel microparticles having a size of greater than about 30 μm, and substantially free of any cells, tissue, or therapeutic compounds, wherein said viscosupplement is configured for introduction into a joint or surgical site of the human or animal subject.

31. A viscosupplement for use according to claim 30, wherein the plurality of porous viscoelastic solid hydrogel microparticles are dispersed in a pharmaceutically-acceptable vehicle.

32. Use of a viscosupplement in treating a joint or surgical site of a human or animal subject such as by lubricating and cushioning intra-articular joint surfaces and tissues, comprising a plurality of porous viscoelastic solid hydrogel microparticles having a size of greater than about 30 μm, and substantially free of any cells, tissue, or therapeutic compounds.
